(19) United States
(12) Patent Application Publication
(10) Pub. No.: US 2017/0156596 A1
(43) Pub. Date: Jun. 8, 2017

(54) CRANIAL IMPLANTS FOR LASER IMAGING AND THERAPY

(71) Applicant: The Regents of the University of California, Oakland, CA (US)

(72) Inventors: Guillermo Aguilar-Mendoza, Corona, CA (US); Javier E. Garay, Riverside, CA (US); Santiago Camacho-Lopez, Ensenada Baja California (MX)

(21) Appl. No.: 15/334,545

(22) Filed: Oct. 26, 2016

Related U.S. Application Data
(63) Continuation-in-part of application No. 14/491,284, filed on Sep. 19, 2014, now Pat. No. 9,535,192.
(60) Provisional application No. 62/247,722, filed on Oct. 28, 2015.
(60) Provisional application No. 61/879,930, filed on Sep. 19, 2013.

(51) Int. Cl. A61B 5/00; A61B 18/02; A61N 5/06

(52) U.S. Cl. CPC A61B 5/0066; A61B 5/0042; A61B 5/4064; A61N 5/0622; A61B 18/0218; A61N 2005/067

(57) ABSTRACT

The disclosure provides for optically transparent cranial implants, and methods of use thereof. In addition, the disclosure provides methods of manufacture for producing, processing, and modifying optically transparent cranial implants.

### CROSS REFERENCE TO RELATED APPLICATIONS

This application claims priority under 35 U.S.C. §119 from Provisional Application Ser. No. 62/247,722, filed Oct. 28, 2015, and is a continuation-in-part of U.S. application Ser. No. 14/491,284, filed Sep. 19, 2014, which application claims priority to Provisional Application Ser. No. 61/879,930, filed Sep. 19, 2013, the disclosures of which are incorporated herein by reference.

### TECHNICAL FIELD

The disclosure provides for cranial implants that are optically transparent so that cellular structures can be visualized by using optical-based diagnostic technology, such as optic coherence tomography. Moreover, the optically transparent cranial implants can also be used in conjunction with low level laser therapy so that the laser light can be administered to brain tissue of shallow or of greater depths.

### BACKGROUND

Since the 1960s, the idea of using laser light to promote therapeutic effects in the brain has been proposed as a therapy to treat various defects and disorders in the brain (Rosomoff et al., Surg Forum, 1965. 16: p. 431-3). However, current methods suffer from high scattering and absorption of IR light, and thus, low energy densities reaching deep targets. Furthermore, laser parameters such as wavelength, pulse duration and irradiation time are very much limited by high absorption and, thus, the heat produced within the scalp, skull and shallow brain tissue. Consequently, the maximum laser dose that may be safely used is limited, thereby hindering the therapeutic effect sought.

### SUMMARY

The disclosure provides for optically transparent cranial implants and procedures using the implants for the delivery of laser light into shallow and/or deep brain tissue. Moreover, the administration of laser light can be used on demand, thus allowing real-time and highly precise visualization and treatment of various pathologies, including but not limited to, traumatic brain injury (TBI) (Mohammed et al., Photomed. Laser Surg. 25(2):107-11 (2007)); Parkinson's disease (Trimmer et al., Uri Oron Molecular Neurodegeneration 4:26 (17 Jun. 2009)); Acute Ischemic Stroke (AIS) (Detaboada et al., Lasers Surg. Med. 38(1):70-3 (2006)); depression (Schiffer et al., Behavioral and Brain Functions 5(46) (200)); and metastatic brain tumor (Rosomoff et al., Surg Forum 16:431-3 (1965).

In a particular embodiment, cranial implants disclosed herein are comprised of an optically transparent material, such as polycrystalline Yttria-Stabilized-Zirconia (YSZ), which can be surgically placed underneath the scalp. In another embodiment, cranial implants disclosed herein further comprise waveguides that are coupled to optical fibers, which allow for the delivery of light, such as laser light, deeply and coherently.

The disclosure provides methods of using the cranial implants disclosed herein. In a particular embodiment, a method using a cranial implant disclosed herein comprises: implanting a cranial implant into a subject; clearing temporally a portion of the scalp in situ; and imaging the targeted brain tissue by using in vivo optical diagnostics technology, such as optic coherence tomography (OCT), which can allow for the imaging of laser-tissue interactions and post-operatory evolution of targeted brain tissue.

The disclosure further provides cranial implants that enable an optically-transparent “window” to the brain, so that the implants may be either permanently or temporarily implanted in the skull, so that the device allows the monitoring and treating a variety of neurological pathologies or traumatic brain injuries. In a certain embodiment, the cranial implants of the disclosure may further comprise additional structural features, such as waveguides and optical fibers, to facilitate the delivery and/or acquisition of light from brain tissue located at greater depths.

The disclosure provides for cranial implants that are comprised of a material that is optically transparent, biocompatible, and has a high refractive index. In another embodiment, a cranial implant disclosed herein is made of a ceramic material, such as Yttrium-stabilized Zirconia. In a further embodiment, a cranial implant disclosed herein further comprises waveguides and is coupled optical fibers. In a particular embodiment, a cranial implant disclosed herein has at least one surface that is rough.

The disclosure also provides for a method of treating a neurological pathology, such as traumatic brain injury, by using Low Level Laser Therapy with a cranial implant disclosed herein.

Additionally, the disclosure provides a method of imaging laser-tissue interactions and post-operatory evolution of brain tissue using a cranial implant disclosed herein with an optical diagnostics technology, such as optic coherence tomography (OCT).

The disclosure provides a method of using the cranial implant disclosed herein, comprising: embedding the implant in a subject's skull; and clearing temporally a portion of the subject's scalp in situ by using optical clearing agents. In a particular embodiment, the optical clearing agents are administered by cryopneumatic technology or by microneedle-based devices.

The disclosure also provides a method for manufacturing the cranial implants disclosed herein, comprising: densifying and annealing nanometric sized ceramic powders into a transparent material; consolidating the densified transparent material into a net shaped form; and/or processing the net shaped form. In another embodiment, the ceramic powder is yttria stabilized zirconia (YSZ) powder having a grain size less than 100 nm, and wherein the powder is annealed at 750° C. in the presence of air for 1 to 15 minutes. In a certain embodiment, the transparent material is produced by using a current-activated powder-assisted densification (CAPAD) based process. In another embodiment, the net shaped form is processed by using pulses from a femtosecond laser with a per-pulse energy between 1 nJ to 8 nJ. In a further embodiment, the laser is used to introduce waveguides and/or change the surface characteristics of the net shaped form.

The disclosure provides for one or more embodiments set forth in the accompanying drawings and the description below. Other features, objects, and advantages of the invention will be apparent from the description and drawings, and from the claims.

### DESCRIPTION OF DRAWINGS

FIG. 1 provides alternate views of a surgically embedded cranial implant of the disclosure in relation to the various tissues found in and around the brain, and the use of a laser in conjunction with the implant.

FIG. 2 provides a schematic diagram representing multiple embodiments of the disclosure, including the manufacture of cranial implants of the disclosure, the processing of the implants, and the use of the implants to image brain tissue.

FIG. 3A-C presents manufactured YSZ materials that by using different reactions conditions generate substantially transparent materials that vary in color from (A) yellow, (B) orange or (C) red.

FIG. 4A-B provides for (A) SEM micrograph of the thermally etched surface of an 8YSZ sample produced via CAPAD at 1200° C. with a 10 min hold time at the temperature, 106 MPa; and (B) photograph of the same 8YSZ on top of backlit text showing transparency of the ceramic.

FIG. 5 provides a schematic of a laser processing set-up to write the waveguide-like structures in transparent ceramic materials.

FIG. 6A-C provides optical micrographs (in transmission) of the waveguide-like structures written in the YSZ ceramic using varying energies: (A) 3.6 nJ per pulse; (B) 4.6 nJ per pulse; and (C) 5 nJ per pulse. The inset number indicates the number of scans along the same track.

FIG. 7 presents a phase contrast micrograph of a waveguide-like structure written at 5 nJ and 200 scans.

FIG. 8A-B provides optical micrographs of waveguide-fiber coupling into a not-annealed sample. (A) Light confinement in a waveguide-like structure written at 5 nJ and 200 scans. (B) Coupling light in a zone out of the waveguide-like structure.

FIG. 9 presents an intensity profile at the output face of the waveguide-like structure for a transmitted beam of a wavelength of 632 nm. The intensity distribution of the waveguide presents two bright spots which could indicate two propagation modes of the waveguide (Inset).

FIG. 10 shows a schematic of the envisioned arrangement of an implant with waveguides and optical fiber configuration.

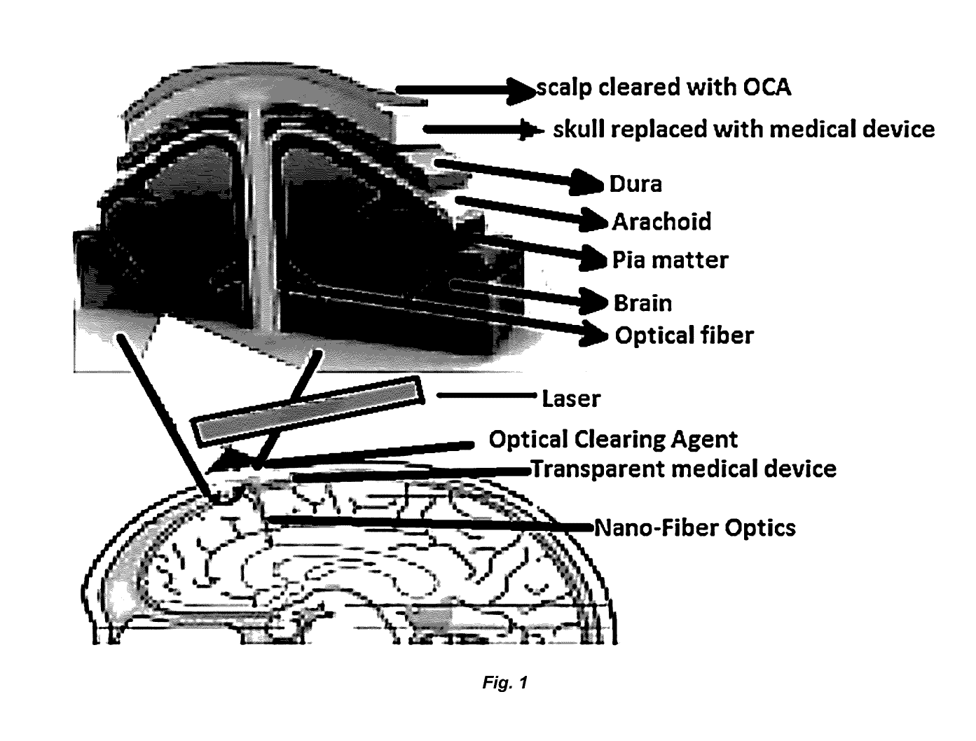

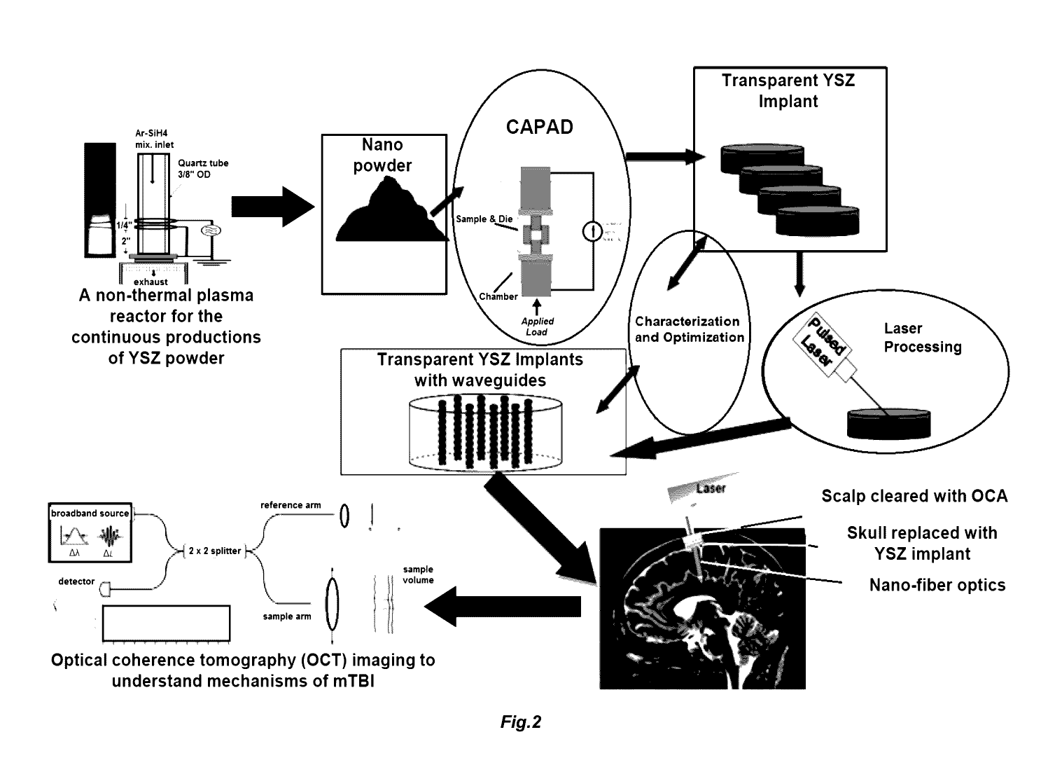

### DETAILED DESCRIPTION

As used herein and in the appended claims, the singular forms “a,” “an,” and “the” include plural referents unless the context clearly dictates otherwise. Thus, for example, reference to “a waveguide” includes a plurality of such waveguides and reference to “the implant” includes reference to one or more implants and equivalents thereof known to those skilled in the art, and so forth.

Unless defined otherwise, all technical and scientific terms used herein have the same meaning as commonly understood to one of ordinary skill in the art to which this disclosure belongs. Although many methods and reagents similar or equivalent to those described herein can be used in the practice of the disclosed methods and compositions, the exemplary methods and materials are now described.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

According to the Defense and Veterans Brain Injury Center (DVBIC), 220,430 service members have been diagnosed with TBI since 2000 within the US military. In the Iraq and Afghanistan conflicts, blast-induced TBI has been identified as “signature injury” caused by the “signature weapon”—the infamous improvised explosive devices or IEDs (D. Wardern., J. Head Trauma Rehabil. 21:398 (2006); and Hoge et al., N. Engl. J. Med. 358:453 (2008)). Moreover, TBI cause 2 million injuries every year in the United States, leading to 56,000 deaths and many of its survivors suffer from long term neurological impairment, costing on the order of $56 billion of direct and indirect costs (Nortje et al., Curr. Opin. Neurol. 17:711 (2004); and Bullock et al., J. Neurotrauma 24 Suppl. 1, 2 p preceding S1 (2007)). Furthermore, based on the World Health Organization (WHO) projection, road traffic accidents, a major cause of traumatic brain injury (TBI), will be the third cause of the global burden of disease and disablement by 2020, behind only ischemic heart disease and unipolar major depression. TBI, accidental or inflicted, is a leading cause of mortality and morbidity in industrial countries among children and young adults. The disclosure provides for a novel and unique optically transparent cranial implant that can be implanted in the cranium, providing a “window” to facilitate the delivery and/or collection of on demand laser light into and from shallow and deep brain tissue. Cranial implants of the disclosure allow for real-time and highly precise visualization and treatment of TBI and other neurological pathologies. The disclosure further provides that cranial implants disclosed herein may be either permanently or temporarily implanted in the skull, and that the cranial implants may further comprise waveguides and optical fibers to facilitate delivery and/or acquisition of light from brain tissue located at greater depths.

Low Level Laser Therapy (LLLT) systems have been developed to treat various neurological pathologies by delivering light through an intact scalp and skull (Detaboada et al., Lasers Surg. Med 38(1):70-3 (2006); Mohammed et al., Photomed Laser Surg 25(2):107-11 (2007); Tegowska et al., Postepy Hig Med Dosw 65:73-92 (2011); Naeser et al., Photomed Laser Surg (2010); and Hashmi et al., PM R 2(12 Suppl 2):S292-305 (2010)). Some of these studies have presented positive results, see e.g., Ying-Ying et al., Low-Level Laser Therapy in Stroke and Central Nervous System, in Handbook of Photonics for Biomedical Science. 2010, CRC Press. p. 717-737. Skepticism remains, however, whether the methods can be adapted to deep brain tissues as the lasers use infrared (IR) light that is prone to high scattering and has limited penetration power due to being high absorbed by the scalp, skull and shallow brain tissue. The absorption of the IR light by these tissues results in unwanted heat production. All of which, result in limiting the applicability and the therapeutic effectiveness of these devices. Cranial implants of the disclosure, however, overcome these drawbacks by providing effective LLLT and other brain therapy to brain tissues at much greater depths than are currently treatable by methods known in the art. Cranial implants of the disclosure also provide a less invasive and potentially more versatile means of delivering light to desired tissues in the brain.

The disclosure provides for cranial implants composed of a hard transparent material. Examples of such materials include but are not limited to, ceramics, plastics, and polymers. In a particular embodiment, a cranial implant disclosed herein is comprised of a transparent ceramic material, such as Yttrium-stabilized Zirconia (YSZ).

The disclosure also provides a method for making cranial implants disclosed herein. The cranial implants of the disclosure can be fabricated by any method known in the art. In a particular embodiment, a method of manufacture for cranial implants disclosed herein use one or more “current-activated powder-assisted densification (CAPAD)” process steps. In a particular embodiment, the CAPAD process is used to densify ceramic powders having powder grain sizes of less than 100 nm, less than 90 nm, less than 80 nm, less than 70 nm, less than 60 nm, less than 55 nm, less than 50 nm, less than 45 nm, less than 40 nm, or less than 35 nm. In a further embodiment, the CAPAD process is used to densify ceramic Yttria stabilized cubic zirconia (YSZ) powders having powder grain sizes of less than 100 nm, less than 90 nm, less than 80 nm, less than 70 nm, less than 60 nm, less than 55 nm, less than 50 nm, less than 45 nm, less than 40 nm, or less than 35 nm.

The disclosure also provides a method for making cranial implants of the disclosure, comprising densifying and annealing materials, such as nanometric powders, with little grain growth, wherein the synergistic combination of very high density and nanoscale sized crystals makes a transparent material. Moreover, the methods provided herein can introduce waveguide-like structures into transparent polycrystalline ceramic materials by using femtosecond laser pulses of a few nJ. As will be further described herein, irradiation using fs laser pulses caused permanent changes in the optical properties of transparent polycrystalline ceramics in the irradiated zone. The laser written structures were found to confine He—Ne laser light (632 nm) thereby introducing waveguide-like structures into the ceramic material. Moreover, waveguide-like structures can be written into transparent ceramic materials, such as YSZ ceramics, using remarkably low per-pulse energy (5 nJ). The number of passes with the laser (i.e., total incident pulses per unit area) was found to affect the waveguide writing. The waveguide-like structures are most likely the result of regions where the concentration of oxygen vacancies and/or their associated free electrons have been altered by laser irradiation.

In a particular embodiment, cranial implants of the disclosure are produced from nanometric powders with narrow-size distribution. In a certain embodiment, cranial implants of the disclosure comprise a grain size of less than 100 nm, less than 90 nm, less than 80 nm, less than 70 nm, less than 60 nm, less than 55 nm, less than 50 nm, less than 45 nm, less than 40 nm, or less than 35 nm. In a further embodiment, the nanometric powders are wide band-gap semiconductor based materials, such as oxides and nitrides. In a certain embodiment, an optically transparent cranial implant of the disclosure will be customized to transmit certain wavelengths, by manufacturing the implant to have a certain color. In another embodiment, an optically transparent cranial implant of the disclosure may have a color between yellow and deep ruby red. In a certain embodiment, a cranial implant of the disclosure is amber in color. In a further embodiment, a cranial implant of the disclosure is substantially clear or colorless.

In a further embodiment, a method for making cranial implants of the disclosure further comprises the step of consolidating the densified transparent material into a net shaped form. Examples of net shaped forms include but are not limited to, substantially circular, oval, rectangular, trapezoidal, polyhedral and square; and wherein the shaped form may be convex, concave or substantially planar. In a further embodiment, the net shaped forms have surfaces that are uniform in texture. In an alternate embodiment, the net shaped forms have surfaces that are not uniform in texture. In particular embodiment, the net shaped form surface or surfaces that will come in contact with osteoblasts and fibroblast will be rough. In another embodiment, the net shaped form surface or surfaces that will do not come in contact with osteoblasts and fibroblast will be substantially polished smooth.

In yet a further embodiment, a method for making cranial implants of the disclosure further comprises processing the net shaped form by using a pulsed laser process to write waveguides into the shaped form or to modify the surface of the net shaped form. In a certain embodiment, the pulsed laser process uses a femtosecond laser. Unexpectedly, the method disclosed herein was found to write waveguides into the net shape form without requiring the use of a relatively high power laser. The unique properties of the densified transparent material generated by using a CAPAD based method can be processed by a laser of surprisingly low power. These unique properties could result from having a high concentration of oxygen vacancies in the material, which can serve as high absorption centers capable of inducing intense interactions that lead to refractive index changes. In a particular embodiment, one or more surfaces of the net shaped form are made rough by using a pulsed laser process.

In an additional embodiment, a method for making cranial implants of the disclosure further comprises attaching micro-optical fibers to the processed shaped form. Micro-optical fibers allow for deeper laser light irradiation, and therefore can provide light to brain tissues of deeper depths.

In a particular embodiment, a cranial implant of the disclosure is comprised of a unique polycrystalline Yttria-Stablized-Zirconia (YSZ) transparent ceramic material, wherein the YSZ material has superior mechanical properties and biocompatible characteristics. In a further embodiment, the YSZ material can be processed to introduce waveguides into the material by using femtosecond laser irradiation using ultralow energies, so as to produce cranial implants of the disclosure having a desired thickness, such as around one millimeter in thickness.

Optogenetics has emerged as a field that combines optics and genetics to control neuronal activity by delivering laser light to specific brain cells. Several efforts are being focused on the design of appropriate approaches to achieve gain or loss of function of neurons expressing specific photosensitive proteins. Potentially, TBI could become one of the brain pathologies that could benefit from optogenetics therapy. However, for this to happen, it is necessary to overcome two inherent TBI problems: (1) highly scattering nature of the scalp which attaches and grows on cranial implants and; (2) the likely existence of multiple affected areas within the brain which must be detected and treated over a prolonged time period, thus limiting the use of optical fibers targeting a single location. The disclosure provides methods for using cranial implants disclosed herein to treat TBI. In a further embodiment, the cranial implants of the disclosure can be used to treat non TBI neurological pathologies. In a particular embodiment, a method for using cranial implants disclosed herein comprises: embedding the cranial implant into the skull of a patient; and then clearing temporally a portion of the subject's scalp in situ by using optical clearing agents (OCAs), such as by using drug perfusion methods, including microneedle injection and cryopneumatic drug delivery. In a particular embodiment, the OCAs are administered to a subject's scalp by using cryopneumatic drug delivery and/or using microneedle devices. In a further embodiment, the method of using a cranial implant disclosed herein further comprises the step of delivering light, such as visible light, to the cranial implant through the scalp, which is then delivered to the targeted brain tissue. Cranial implants of the disclosure can deliver light to shallow brain targets, or if the cranial implants further comprise waveguides coupled to optical fibers can deliver light to much deep brain tissue targets.

The disclosure describes the development of a bio-ceramic material that allows for delivery of laser light into shallow and deep tissue on demand, thus allowing real-time and highly precise visualization and treatment of various pathologies (e.g., traumatic brain injury (TBI), stroke, Parkinson's disease, Acute Ischemic Stroke (AIS), depression, metastatic tumors, reconstructive shoulder or knee surgery, etc.) The disclosure describes this development using optically transparent ceramic implants made of nano-crystalline Yttria-Stabilized-Zirconia (nc-YSZ) or Alumina (Al2O3), which may be surgically placed subcutaneously—some with waveguides coupled to optical fibers to deliver laser light deeply and coherently for in vivo optical diagnostics technology, such as optic coherence tomography (OCT), for the imaging of laser-tissue interactions, and/or post-operatory evolution of targeted tissue. In short, the disclosure describes devices and methods for an optically-transparent material that may be either permanently or temporarily implanted subcutaneously and potentially instrumented with waveguides and optical fibers to deliver and/or acquire light from either shallow or deep tissue targets to monitor and treat a variety of pathologies or injuries.

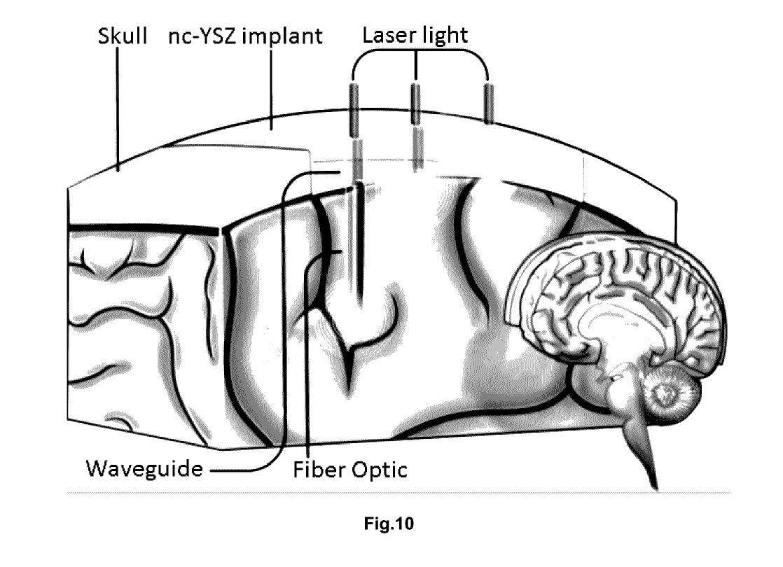

The idea of using laser light to promote therapeutic effects in the brain has been proposed and discussed. Although a number of materials (e.g., titanium, alumina, and acrylic) have been evaluated for use for many medical prosthesis, none have previously provided the requisite combination of transparency and toughness required for clinically-viable prosthetics. The disclosure shows that nc-YSZ offers an attractive alternative as a prosthesis due to its unique transparency, as well as the proven biocompatibility of YSZ in dental and orthopedic applications. In the short term and regardless of the medical application, many laboratory and regulatory tests are required for a myriad of medical treatments (whether they include implants or not). The development of a ceramic biomaterial platform will allow for in vivo pre-clinical long-term studies for which optical access is essential to assess biocompatibility and efficacy. Moreover, the goal is much more ambitious. Due to scattering, any irradiated light shone into tissue or collected from bioluminescent molecules is unlikely to travel too far, thus limiting the interrogation/therapeutic field to the near-field region. Fortunately, the optical properties of nc-YSZ implants allow for the use of femtosecond (fs) laser irradiation to post-process emissions through the transparent implants and inscribe waveguides across them (see FIG. 10), enabling deeper coherent light delivery for mid-range access. Furthermore, with the addition of fiber optics, which are planned to couple with waveguides written across the nc-YSZ implants (see FIG. 10) one could enable light-tissue interactions within the realm of photo-biomodulation, which is important for the potential chronic therapy of many neural disorders. Thus, this platform could be designed for simultaneous shallow, mid, and deep tissue accessibility. In its final form, this platform allows: a) direct, real-time, long-term monitoring of imaging biomarkers associated with chronic traumatic injuries, allowing tracking of changes currently only measurable by post mortem histology; b) chronic optical monitoring and/or more precisely targeted photodynamic treatments of residual gliomas following tumor excision; c) non-invasive laser ablation therapies without recurring surgeries; and d) novel modalities of optical neuromodulation for clinical applications from neurology to psychiatry.

The fabrication of the YSZ implants can utilize a method known as “current-activated powder assisted densification (CAPAD)”, a processing method that simultaneously applies high current densities and pressures in addition to the traditional processing parameters of temperature and time. The current serves as the method's sole source of heat (in the form of Joule heating). Although the current is the distinguishing feature, it should be emphasized that it is the complimentary contributions of current and an applied pressure that make it successful. The high current flux not only results in very high heating and cooling rates, but also produces good temperature homogeneity. The applied pressure aids the densification process by increasing the surface energy driving force and affects the densification mechanism. Due to the combination of the current effects and applied pressure, the technique described has proven effective in significantly lowering the processing temperature required for consolidating ceramics to full density. Because decreasing processing temperature and time is crucial to the retention of a nanocrystalline microstructure, the CAPAD method is ideal for fabricating nanoceramics.

The grain boundary diffusivity of zirconia is influenced by the electric current in the process. The disclosure shows that a two-step pressure ramp is useful for retaining nanostructured grains in fully dense materials. The disclosure shows the ability to densify the materials with remarkably little grain growth. The initial grain size of the commercial powder was 50 nm which is the same as the dense samples. The combination of very high density and nanoscale crystals makes the materials transparent. Interestingly the sample also has an amber color. The color of the samples can be readily changed from a yellow to deep ruby red.

Once the material is consolidated in net shape form, a pulsed laser processing is used, which is an established method for writing waveguides in optical materials. Waveguides in optical media are an essential part of a wide variety of important optical devices. In recent years, pulsed laser processing has become an established method for writing waveguides in optical materials; successful examples using femtosecond (fs) laser pulses exist for glasses and single crystals. Transparent polycrystalline ceramics have been receiving significant increased attention for optical applications such as laser source materials, solid state lighting and light manipulation. Ceramics offer high temperature and chemical stability and relatively efficient fabrication compared to glasses and single crystals thus promising to increase the application space for optical materials. A potential drawback (especially in the ceramic case) is the relatively high power necessary for the successful writing if the waveguides, but preliminary data indicates that the high concentration of oxygen vacancies in the YSZ samples already manufactured serve as high absorption centers capable of inducing intense interactions that lead to refractive index changes. For the laser processing of the ceramics, a femtosecond laser will be used. Once waveguides are written on the ceramics we will explore the possibility of attaching micro-optical fibers for deeper laser light irradiation. Herein is presented a method for writing waveguides in transparent polycrystalline ceramics using fs laser pulses with remarkably low energy. The low energy requirements for writing waveguides should make these optical ceramics more amenable for use as cranial implants.

In the experiments presented herein, by using ultralow energy (e.g., 5 nJ) and ultrafast (e.g., 66 fs) laser pulses waveguide-like structures were introduced into transparent polycrystalline ceramic materials. The energy level of the laser pulses used in writing these waveguide-like structures is at least three or four orders of magnitude lower than previously reported for ceramics, and are the lowest energies ever reported for the successful writing of waveguides in a ceramic material. Further, it was found that the refractive index change increases with the pulse energy and the number of scans. Moreover, it was also found that the writing of these structures is easier for samples with a low annealing time (high linear absorption coefficient).

The disclosure also provides for making transparent polycrystalline ceramic materials disclosed herein. In a particular embodiment, the transparent ceramic materials can be manufactured by utilizing one or more current-activated powder-assisted densification (CAPAD) process steps. The CAPAD process for making the transparent polycrystalline ceramic materials includes the step of densifying and annealing the ceramic materials, such as nanometric powders, with little grain growth, wherein the synergistic combination of very high density and nanoscale sized crystals makes a transparent material. Typically, the transparent ceramic materials are produced from nanometric powders with narrow-size distribution. In a further embodiment, the CAPAD process is used to densify ceramic powders having powder grain sizes of less than 100 nm, less than 90 nm, less than 80 nm, less than 70 nm, less than 60 nm, less than 55 nm, less than 50 nm, less than 45 nm, less than 40 nm, or less than 35 nm. In yet a further embodiment, the CAPAD process is used to densify ceramic Yttria stabilized cubic zirconia (YSZ) powders having powder grain sizes of less than 100 nm, less than 90 nm, less than 80 nm, less than 70 nm, less than 60 nm, less than 55 nm, less than 50 nm, less than 45 nm, less than 40 nm, or less than 35 nm.

As the transmission of light through a sample is related to the average concentration of oxygen vacancies, there is an increase in transmission when the samples are annealed for various times in the presence of oxygen (e.g., air) at an elevated temperature (e.g., 750 CC). In a particular embodiment, the transparent ceramic material is annealed at an elevated temperature in the presence of oxygen for at least 1, 2, 3, 4, 5, 6, 7, 8, 9, 10, 11, 12, 13, 14, 15, 20, 30, 40, 60, 90, 120, 180, 240, 360, or 480 minutes. In a further embodiment, the transparent ceramic material is annealed at an elevated temperature (e.g., 750 CC) in the presence of oxygen (e.g., air) for at least 1 to 15 minutes. In alternate embodiment, the transparent ceramic material is not annealed prior to processing with femtosecond laser pulses.

The disclosure further provides that the transparent polycrystalline ceramic materials may be customized to transmit certain wavelengths, by manufacturing the materials to have a certain color. In another embodiment, the ceramic materials disclosed herein may have a color between yellow and deep ruby red. In a further embodiment, the ceramic materials disclosed herein may be substantially clear or colorless.

In a further embodiment, a method for making the transparent polycrystalline ceramic materials of the disclosure comprises the step of consolidating the densified transparent material into a net shaped form. Examples of net shaped forms include but are not limited to, substantially circular, oval, rectangular, trapezoidal, polyhedral and square; and wherein the shaped form may be convex, concave or substantially planar. In a further embodiment, the net shaped forms have surfaces that are uniform in texture. In an alternate embodiment, the net shaped forms have surfaces that are not uniform in texture.

The disclosure provides for writing or introducing waveguide like structures into the transparent polycrystalline ceramic materials by using a pulsed laser process. In experiments presented herein, it was found that waveguide-like structures could be introduced into the transparent ceramic materials by using an unexpectedly low power laser, such as a femtosecond laser. In a particular embodiment, waveguide-like structures can be introduced/written into transparent polycrystalline ceramic materials by using femtosecond laser pulses having per-pulse energy between 1 nJ to 15 nJ, 1 nJ to 12 nJ, 1 nJ to 10 nJ, 1 nJ to 8 nJ, 1 nJ to 7 nJ, 1 to 6 nJ, 1 to 5 nJ, 2 to 4 nJ. In another embodiment, waveguide-like structures can be introduced/written into transparent polycrystalline ceramic materials by using femtosecond laser pulses having per-pulse energy of about 3 nJ, 3.1 nJ, 3.2 nJ, 3.3 nJ, 3.4 nJ, 3.5 nJ, 3.6 nJ, 3.7 nJ, 3.8 nJ, 3.9 nJ, 4 nJ, 4.1 nJ, 4.2 nJ, 4.3 nJ, 4.4 nJ, 4.5 nJ, 4.6 nJ, 4.7 nJ, 4.8 nJ, 4.9 nJ, 5 nJ, 5.1 nJ, 5.2 nJ, 5.3 nJ, 5.4 nJ or 5.5 nJ. Moreover, in experiments presented herein it was also found that the number of incident pulses per unit area at each energy level played an unexpected role in shaping/demarking the waveguide like structures. In particular, it was found that no matter the per-pulse energy level, as the scan number increased the waveguide-like structural changes to the ceramic material became more and more apparent. Accordingly, the disclosure provides for fine tuning the demarcation of waveguide-like structures in transparent ceramic materials by varying the number of incident pulses per unit area. In a certain embodiment, waveguide-like structures can be introduced/written into transparent polycrystalline ceramic materials by using from 100 to 300, 110 to 280, 120 to 260, 130 to 240, 140 to 220, or 150 to 210 incident pulses per unit area. In a further embodiment, waveguide-like structures can be introduced/written into transparent polycrystalline ceramic materials by using about 100, 110, 120, 130, 140, 150, 160, 170, 180, 190, 200, 210, 220, 230, 240, or 250 incident pulses per unit area. It should be further understood that the foregoing embodiments directed to the incident pulses per unit area can be all be performed with the same per-pulse energy or alternatively where some pulses are performed at certain per-pulse energy while others are performed at different per-pulse energies.

The following examples are intended to illustrate but not limit the disclosure. While they are typical of those that might be used, other procedures known to those skilled in the art may alternatively be used.

### Examples

CAPAD Process to Make Densified Transparent Materials.

To fabricate cranial implants of the disclosure, a current-activated powder-assisted densification (CAPAD) process can be used. CAPAD is a processing method that simultaneously applies high current densities and pressures in addition to the traditional processing parameters of temperature and time. The current serves as the method's sole source of heat (in the form of Joule heating). Although the current for CAPAD is the distinguishing feature, it should be emphasized that it is the synergistic contributions of current and an applied pressure which make it a useful process. The high current flux not only results in very high heating and cooling rates, but also produces good temperature homogeneity. The applied pressure aids the densification process by increasing the surface energy driving force and affects the densification mechanism (Coble, R. L., J. Appl. Phys. 85:4798 (1970)). Due to the combination of the current effects and applied pressure, the technique is effective in significantly lowering the processing temperature required for consolidating materials, such as ceramics, to full density (Groza et al., Rev. Adv. Mat. Sci., 2003. 5:24-33 (2003)). Because decreasing processing temperature and time is crucial to the retention of a nanocrystalline microstructure, the CAPAD process is ideal for fabricating nanoceramics.

Manufacturing Yttrium-Stabilized Zirconia (YSZ)-Based Cranial Implants Using the CAPAD Method:

Polycrystalline Yttrium-stabilized Zirconia (YSZ) ceramics can provide unmatched optical properties. Based on the composition, and controlling the sintering and annealing condition, YSZ ceramics can be manufactured so that the material is transparent with a high refractive index, which further allows the transmission of light having a wavelength between UV and the near-IR spectrum. These properties make YSZ-based materials suitable for optical coatings and wave-guided imaging (Lange et al., Journal of Non-Crystalline Solids 354:4380 (2008)). Moreover, YSZ materials have superior mechanical properties, such as high hardness and toughness (more than 3 times tougher than optical glasses, and excellent thermal insulating characteristic (10 times better than aluminum oxide) (Casolco et al., Scripta. Mater 58:516 (2008); and Ghosh et al., J. Appl. Phys. 106 (2009)).

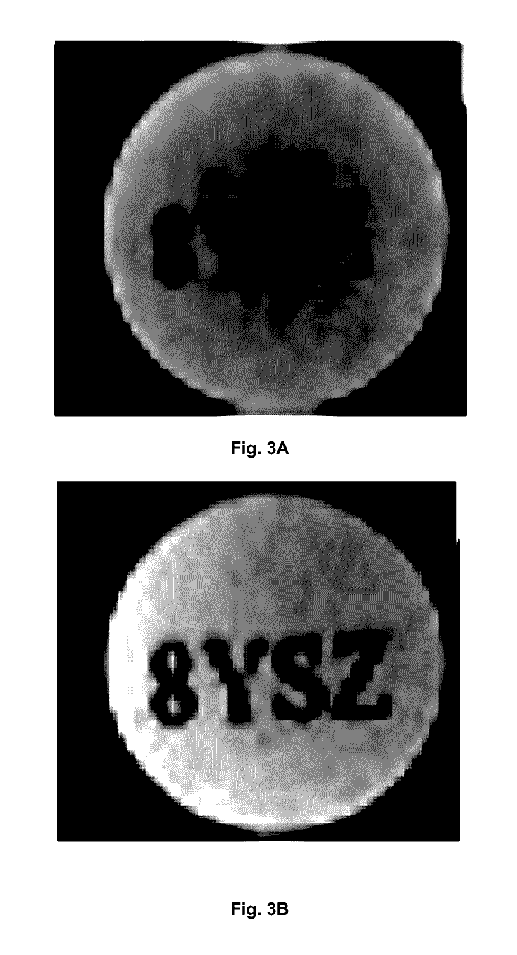

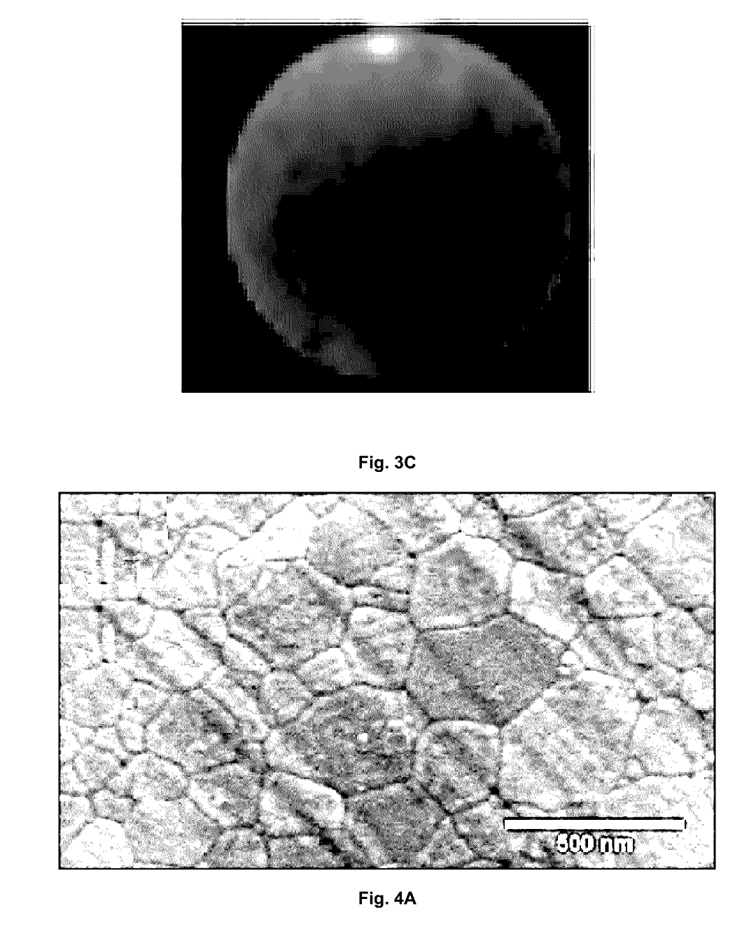

The grain boundary diffusivity of zirconia is influenced by the electric current in the CAPAD process. Cranial implants comprising YSZ materials were made with a two-step pressure ramp, using Yttria stabilized cubic zirconia (YSZ) ceramic powders having particles around 50 nm in size. The reaction conditions generated transparent YSZ-based materials that had both a high density and a nanocrystalline microstructure that was largely retained through the entire manufacturing process. Based on the reaction conditions, the color of the resulting YSZ materials can be varied (see e.g., FIG. 3).

Ceramic Fabrication.

Commercial (Tosoh Corporation, Tokyo, Japan) nanocrystalline 8YSZ powder with a reported grain size of 50 nm was densified using current activated pressure assisted densification (CAPAD). Each of the samples was prepared in a graphite die with 19 mm inner diameter. Temperature was measured using a grounded N-type thermocouple placed in a hole drilled halfway through the thickness of the die. The CAPAD processing was done in a custom built apparatus as described in Casolco et al. (Script. Mater. 58:516-519 (2008)). 8YSZ powder (1.5 g) was loaded into the die for each sample. The pressure in the system was raised to 106 MPa before the current was applied.

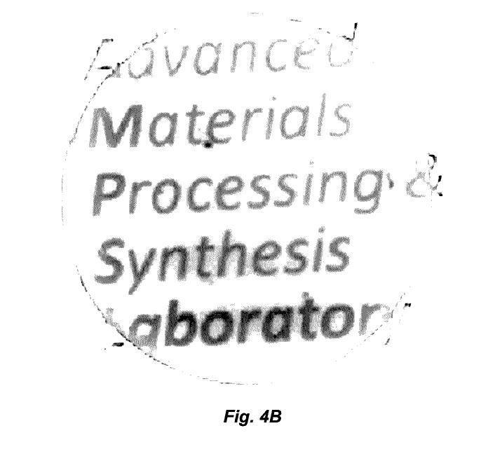

When this pressure was reached, the current heated the sample to 1200° C. using a heating rate of 200° C./min. Samples were held for 10 min at final pressure and temperature. The samples were mechanically polished for microstructural and optical characterization. The densified samples were fractured and examined using scanning electron microscopy (SEM) (Phillips FEI). A SEM micrograph and optical photograph of a typical sample is presented in FIG. 4. The sample was annealed for 8 hours in air at 750° C., in order to thermally etch the surface.

Ceramic Annealing.

The optical properties, such as absorption coefficient, are highly dependent on oxygen stoichiometry in 8YSZ. Oxygen stoichiometry in YSZ can be readily controlled by exposure to either reducing or oxidizing atmospheres. The as-processed ceramics presented herein are oxygenic deficient due to the reducing nature of CAPAD processing conditions. However, by annealing the YSZ samples in air, results in the diffusion of oxygen back into the sample and a reduction in the number and size of oxygen vacancies that were created via the high temperature heating. Thus, as the transmission of light through a sample is related to the average concentration of oxygen vacancies, there is an increase in transmission when the samples are annealed for various times. In order to evaluate the effects of annealing on waveguide writing, the samples were annealed in air at 750° C. for various times (10 min to 8 h).

Processing YSZ-Based Cranial Implants by Writing Waveguides Across its Thickness with Low Energies.

Ultralow energies, approximately 3 nJ, were sufficient to trace waveguides with femtosecond lasers in the YSZ materials. Further optimization of the composition of the YSZ materials and the laser processing parameters produces high quality waveguides with even longer laser pulses (pico or even nanosecond) reducing complexity and manufacturing costs. Then a femtosecond laser pulse is focused inside a transparent material the irradiance reached in the focal volume may induce nonlinear absorption through a combination of multiphoton absorption, avalanche ionization and tunneling ionization. If enough energy is deposited in the material through this nonlinear absorption, permanent structural changes such as refractive index changes can be induced. Since the nonlinear absorption can be only reached for high irradiances, the changes are induced only in the focal volume. Therefore, by simply translating the sample with respect to the focus and using a continuous train of pulses it is possible to induce permanent changes in the material in a reproducible and controlled manner. This technique was used to write waveguide-like structures into an YSZ ceramic material.

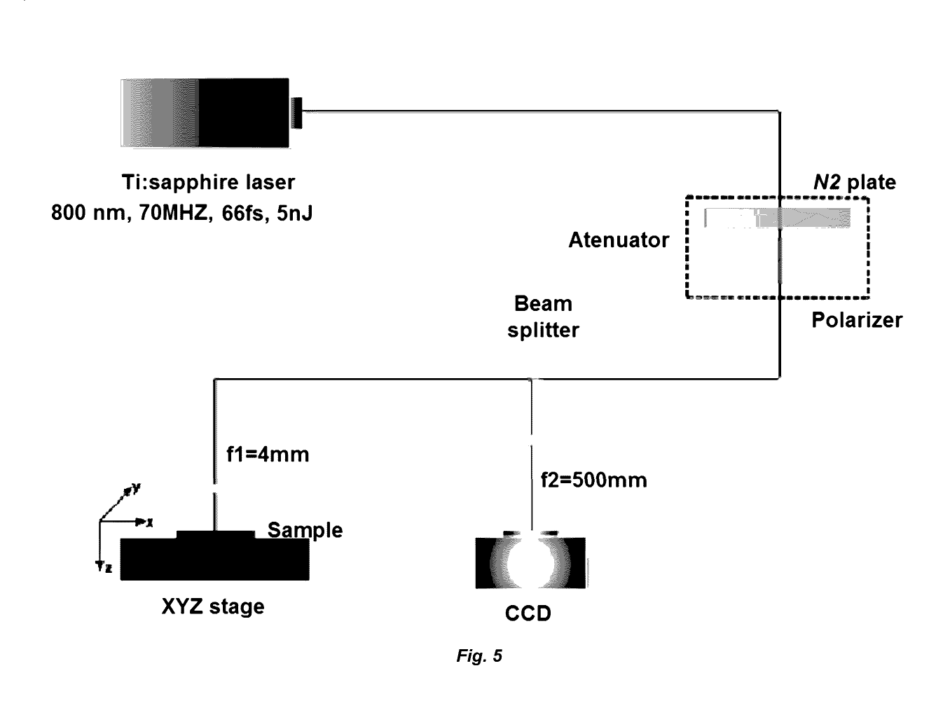

For the laser processing of the ceramics a custom Ti: sapphire oscillator with a central wavelength of 800 nm, a repetition rate of 70 MHz, a maximum on-target per pulse energy of 5 nJ and pulse duration (FWHM) of about 66 fs was used. The laser beam was focused on the surface sample through a lens of 0.6 N.A. and a focal distance f₁=4 mm. To find and visualize the beam waist onto the sample (ω₀=1.9 μm, z_{R}=7.8 μm a lens of focal distance f₂=500 mm and a CCD camera (PL-A774, Pixelink) was used. This system was set in a configuration similar to an image relay arrangement. The samples were translated with a constant speed of 530 μm/s and perpendicular to the incident beam (e.g., see schematic in FIG. 5). To determine the refractive index change threshold, a series of about 2 mm long lines were written varying the number of laser scans and the per pulse delivered energy by using an attenuator made of a combination a half-wave plate and a polarizer.

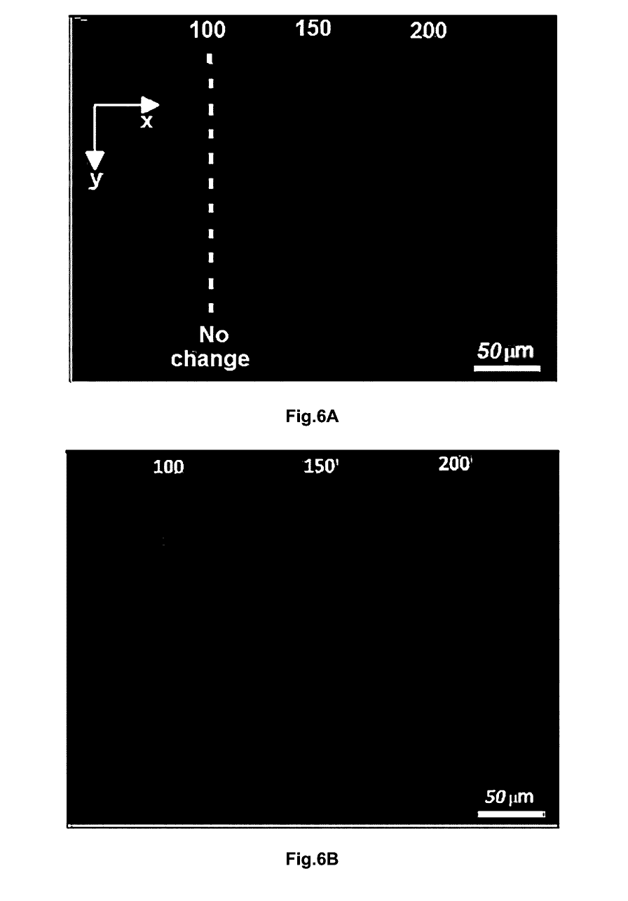

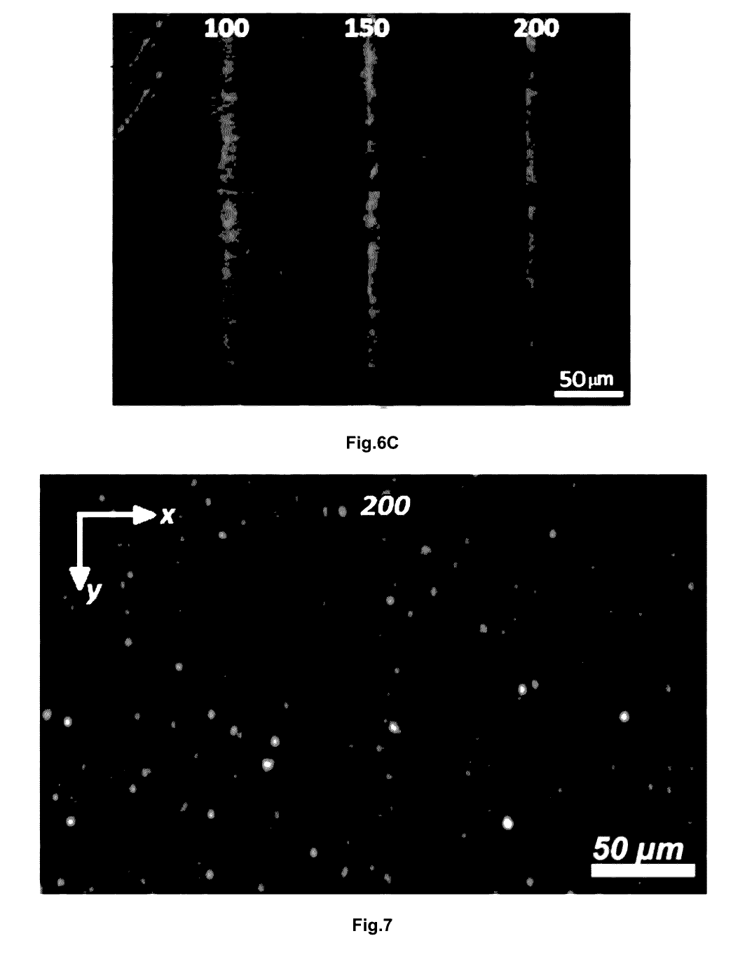

A top view optical micrograph of a series of waveguide-like structures on as processed YSZ is shown in FIG. 6. The micrographs reveal that with each of the laser energies in this study, pulsed laser processing produced written structures that were brighter compared to the surrounding material, indicating a permanent change in the optical properties of the irradiated region. This color change can be caused by a change in absorption coefficient, refractive index or both as will be discussed further herein.

The energies used for laser irradiation (3.6 nJ to 5 nJ) are at least three or four orders of magnitude lower than previously reported in the literature for laser-induced index trimming in transparent crystals and ceramics. The lowest (threshold energy) for writing is 3.6 nJ (I=4.4×10¹¹ W/cm², F=31 mJ/cm²). In order to verify that the energy was below the ablation threshold, the irradiated zones were imaged using a scanning electron microscope (SEM). There was no evidence of ablation.

The number of scans was also found to play an important role in waveguide writing at the energies tested. For example, at 3.6 nJ (see FIG. 6A) at a low number of scans (100) no visible change was observed, while increasing the number of scans to 150 produced a weak visible change, and at 200 scans the wave-guide structure were clearly visible. The effects of scans were similar at higher energies (see FIG. 6B-C). The dependence on the total number of pulses indicates that the total incident pulse per unit area is an important aspect in forming the wave-guide structures.

Changes in the optical properties during fs processing are often attributed to laser-induced structural changes due to the heat accumulation and field or irradiance related effects over the irradiated zone. It was not expected, however, that such phase changes would occur in the samples tested, since fully stabilized YSZ has a cubic structure that is very stable. It was postulated that the observed changes in the materials are related to point defects. Previously, it was found YSZ could be induced to change color by changing the oxygen stoichiometry in the sample by annealing in the presence of oxygen. The primary absorption centers in YSZ are oxygen vacancies, V_{ö} with trapped electrons, e′ producing oxygen vacancies with a single positive charge written in Kroger-Vink notation as:

V_{ö}+e′→V_{ö} (1)

By annealing in the presence of oxygen one can change the concentration of oxygen vacancies with trapped electrons, [V_{ö}]. The color change induced by fs laser processing induces a similar change (i.e. changes in the concentration of oxygen vacancies with single positive charge, [V_{ö}]). In addition to the color changes, the oxygen stoichiometry changes in YSZ can also affect the refractive index.

Equation (1) suggest that there are two possibilities for controlling [V_{ö}]: (1) The concentration of [V_{ö}] can vary or (2) the trapped electron e′, can become de-coupled from the [V_{ö}]. Both mechanisms are feasible with pulsed laser processing. Mechanism (1) requires thermally driven diffusion of oxygen vacancies. fs laser irradiation could increase the temperature of the samples to temperatures where oxygen vacancies have sufficient mobility to cause significant diffusion. This is feasible because of the time between successive delivered pulses (14 ns, i.e. 70 MHz repetition rate), which for this material, could be much shorter than the characteristic time for thermal diffusion out of the focal volume. As a result, the delivered train of laser pulses deposits energy faster than the time required for heat diffusion to occur, leading to a high temperature rise of the material over the focal region. For long enough laser exposures, the heat deposited by the successive pulses of the oscillator diffuses towards the surrounding material inducing changes beyond the focal volume. This can be seen in FIG. 3C, where the width of the structures is larger than the diameter of the laser spot 4 μm). It is possible to reach high temperatures via heat accumulation processes by using a high-repetition rate femtosecond laser.

It is also possible that high electric field caused by the fs laser interaction with YSZ causes decoupling of electrons trapped in vacancies (Mechanism (2)). Two types of oxygen vacancies have been identified in reduced cubic YSZ: T- and C-Type oxygen vacancies. The T-type vacancies occur in weakly reducing conditions, while the C-Type occurs in strongly reducing atmospheres. The energy gaps between the T- and C-Type oxygen vacancies and the conduction band in reduced YSZ are ˜3.3 eV and ˜2.6 eV, respectively. As the samples are irradiated with A=800 nm, corresponding to a photon energy of Eₚₕ=1.55 eV, it is apparently not possible to induce changes in the YSZ via Mechanism 2 with linear photonic absorption. However, since fs-laser pulses are employed, the intensities are very high indicating the possibility of non-linear effects, in particular 2-photon absorption. The doublet of Eₚₕ is 3.10 eV which is enough energy to decouple trapped electrons from the C-type oxygen vacancies via Mechanism 2.

In order to determine whether the induced color change in the irradiated zones also led to a refractive index change, the post-laser processed ceramics were analyzed using a phase contrast microscope (Olympus, model BX41). FIG. 7 shows a phase contrast micrograph of a waveguide-like structure fabricated at 5 nJ per pulse energy (F=44 mJ/cm², I=6.7×10¹¹ W/cm²) and 200 scans. The micrograph reveals a high contrast between the resulting structure and the surrounding material. This clearly indicates a refractive index change over the irradiated track. The bright spots visible in sample are likely dust particles and/or other imperfections caused by polishing.

While the color change in YSZ has been associated with variance in the oxygen vacancy concentration, permittivity experiments showing that the refractive index is also coupled to the degree of oxygen reduction were performed. There is a clear inverse relation between the oxygen vacancy concentration and the relative permittivity in YSZ.

Measurements were performed at significantly lower than optical frequencies (10³ Hz), but presumably similar changes could occur over a wide frequency range. Since the refractive index is related to the permittivity by:

$$n ∝ {( \frac{ɛ}{{ɛ}_{0}} )}^{\frac{1}{2}} \qquad ( 2 )$$

where n is the refractive index, ε and εₒ are the relative and free space permittivities respectively, it also follows that there is an inverse relation between the oxygen vacancy concentration and the refractive index. This means that n should increase as Vₒ decreases. Thus, it is postulated that the waveguide-like structures are caused by a change in fs-laser irradiation decreasing the oxygen vacancy concentration thereby increasing the refractive index.

The irradiation results for samples with different annealing times (i.e., different optical properties) are presented in TABLE 1. All the results were obtained for a per pulse energy of 5 nJ (F=44 mJ/cm², I=6.7×10¹¹ W/cm²) and 200 scans.

TABLE 1
Laser induced changes in the YSZ ceramic
as a function of the annealing time.

| | Transmittance (%) | |
|---|---|---|
| Annealing time | at 800 nm | Induced change |
| Not annealed | 15 | Waveguide-like |
| | | structure |
| 10 min hold | 14 | Waveguide-like |
| | | structure |
| 15 min hold | 13 | No change induced |
| 30 min hold | 20 | No change induced |
| 45 min hold | 20 | No change induced |
| 1 h hold | 28 | No change Induced |
| 8 h hold | 38 | No change induced |

It was found that the waveguide-like structures writing is easier for samples with lower annealing times that have lower optical transmittance (higher linear absorption coefficient). This is further evidence that the waveguide writing process is an analogous effect to annealing (i.e., is dependent on the concentration of oxygen vacancies in the sample [V_{ö}]).

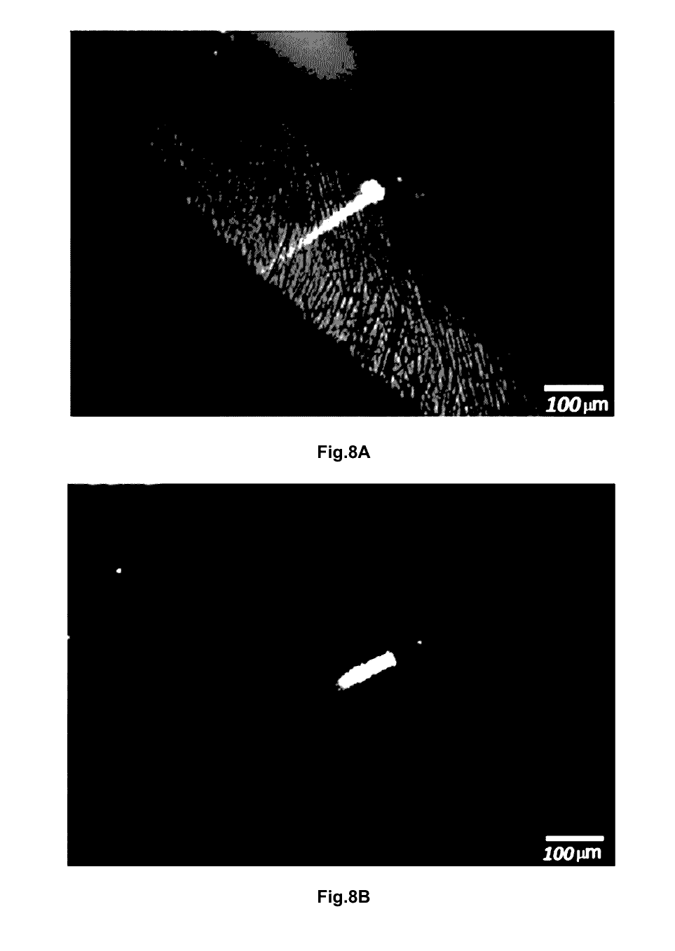

The ability of the written structures to behave as an optical waveguide was proven by coupling a He—Ne laser through a single mode optical fiber. The output of the waveguide was collected by a 10× microscope objective coupled to a CCD camera. The waveguide-fiber coupling into a not-annealed sample is presented in FIG. 8. It was seen that there is indeed light confinement in the written structures. These results also indicated that the change induced over the irradiated zone corresponds to a refractive index increment (positive Δn). It was also found that the structures written at higher energies present a better light confinement than those written at lower energies. It was further observed that better light confinement was achieved in structures when written with a higher number of scans. This confirms a higher refractive index increment over the irradiated zone for structures written at higher energies and higher number of scans.

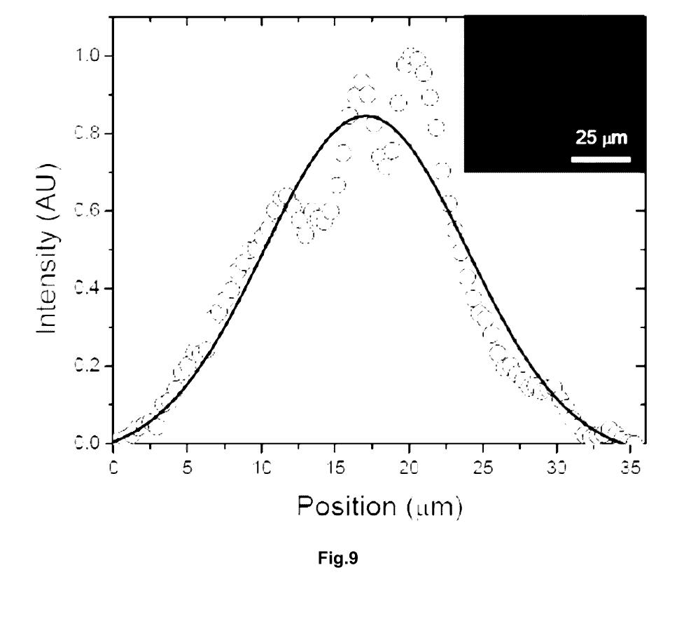

The intensity profile at the output face of a waveguide written using 5 nJ (F=44 mJ/cm², I=6.7×10¹¹ W/cm²) and 200 scans is shown in FIG. 9. The waveguide does not produce a single mode intensity distribution at the coupling wavelength (632 nm). This is shown in the inset picture of FIG. 9, which shows the mode intensity distribution of the waveguide. Instead, it presents two bright spots which could be two propagation modes of the waveguide. Another important point to note is that the transmittance of the YSZ ceramics increases with increasing wavelengths, suggesting that the transmittance of the waveguide structures should be higher at higher wavelengths.

In Vitro Studies to Determine Optimal Roughness of YSZ for Appropriate Cell Response.

Osteoblast adhesion and long-term functions are key factors in determining the integration of skull and the ceramic implants, with minimal use of adhesives. In vitro studies are performed to determine optimal surface roughness for cranial implants for favorable osteoblast and fibroblast interactions. Various changes in mouse fibroblast and osteoblast cells morphology, and cellular functions will be monitored, including cell viability, adhesion, morphology, proliferation, and long term functions.

Cryopneumatic Technology and Microneedle Devices to Deliver Optical Clearing Agents:

Cryopneumatic technology (CPx) can significantly enhance percutaneous drug delivery (PDD) of high molecular weight compounds (Aguilar et al., J Drugs Dermatol 9:1528 (2010); Cilip et al., J Biomed Opt 15:048001 (2010); and Cilip et al., Lasers Surg Med 41:203 (2009)). Hollow microneedle injection is effective in enhancing percutaneous drug delivery (Hoge et al., N Engl J Med 358:453 (2008); and J. Yoon et al., Enhancement of optical skin clearing efficacy using a microneedle roller. (SPIE, 2008), vol. 13, pp. 021103.). CPx and microneedle devices are used to deliver optical clearing agents (OCAs) into the scalp to increase its transparency and allow for easier light transmittance to cranial implants of the disclosure. Using the CPx and microneedle devices to administer OCAs eliminates the need of having to constantly remove the scalp or having to fix permanent channels/fibers through it.

Ex Vivo and In Vitro Models of Delivering OCAs to the Scalp:

OCAs are delivered in in vitro and ex vivo mouse skin models and cell cultures grown on YSZ substrates by using CPx or microneedle devices. From which, optimal penetration rate and depth for administering the OCAs using CPx or microneedle injection is determined. In addition, the clearing potential of the OCAs over time is determined by measuring the reflectance and transmittance of administered light. In the ex vivo model, a perfusion bed system is used to replicate in vivo subdermal blood perfusion. The rate and depth of drug penetration is then determined.

OCT Imaging with Cranial Implants.

Studies to assess the extent to which OCT visualization is improved by using transparent implants of the disclosure are performed. OCT imaging with the cranial implants are evaluated based upon axial and lateral resolution and penetration depth into the brain. Longer term observation of brain injury before and during laser therapy is evaluated by using OCT with the cranial implants. OCT data is acquired and processed to systematically study the time course of optically derived measures of TBI versus those from intracranial pressure measurements and clinical observation.

A number of embodiments of the invention have been described. Nevertheless, it will be understood that various modifications may be made without departing from the spirit and scope of the invention. Accordingly, other embodiments are within the scope of the following claims.

1. A cranial implant comprising a material that is optically transparent, biocompatible, and has a high refractive index.

2. The cranial implant of claim 1, wherein the material is ceramic.

3. The cranial implant of claim 2, wherein the material is Yttrium-stabilized Zirconia (YSZ).

4. The cranial implant of claim 1, wherein the implant further comprises waveguide-like structures and is coupled optical fibers.

5. The cranial implant of claim 1, wherein at least one surface of the implant is rough.

6. A method of treating a neurological pathology by using Low Level Laser Therapy with the cranial implant of claim 1.

7. The method of claim 6, where the neurological pathology is traumatic brain injury.

8. A method of imaging laser-tissue interactions and post-operatory evolution of brain tissue using the cranial implant of claim 1 with optical diagnostics technology.

9. The method of claim 8, wherein the optical diagnostics technology is optic coherence tomography (OCT).

10. The method of claim 6, wherein the method comprises:
(a) embedding the implant in a subject's skull; and
(b) clearing temporally a portion of the subject's scalp in situ by using optical clearing agents.

11. The method of claim 10, wherein optical clearing agents are administered by cryopneumatic technology or by microneedle-based devices.

12. A method of manufacturing the cranial implants of claim 2, comprising:
densifying and annealing nanometric sized ceramic powders into a transparent material; and
consolidating the densified transparent material into a net shaped form.

13. The method of claim 12, wherein the ceramic powder is yttria stabilized zirconia (YSZ) powder having a grain size less than 100 nm, and wherein the powder is annealed at 750° C. in the presence of air for 1 to 15 minutes.

14. The method of claim 12, wherein the transparent material is produced by using a current-activated powder-assisted densification (CAPAD) based process.

15. The method of claim 14, further comprising
processing the net shaped form.

16. The method of claim 15, wherein the processing is performed using pulses from a femtosecond laser with a per-pulse energy between 1 nJ to 8 nJ.

17. The method of claim 16, wherein the femtosecond laser is used to introduce waveguides and/or change the surface characteristics of the net shaped form.
